(19) United States
(12) Patent Application Publication
(10) Pub. No.: US 2004/0072201 A1
(43) Pub. Date: Apr. 15, 2004

(54) Methods of identifying compounds that inhibit nonstop degradation of mRNA

(76) Inventors: Harry C. Dietz, Towson, MD (US); Pamela A. Frischmeyer, Baltimore, MD (US); Roy Robert Parker, Tucson, AZ (US); Ambrosius Theodorus Cornelis Van Hoof, Pearland, TX (US)

Correspondence Address: Jennifer K. Rosenfield, Intellectual Property Practice Group, Edwards & Angell, LLP, P.O. Box 9169, Boston, MA, 02209, US

(21) Appl. No.: 10/417,354

(22) Filed: Apr. 16, 2003

Related U.S. Application Data
(60) Provisional application No. 60/373,093, filed on Apr. 16, 2002.

(51) Int. Cl. C12Q 1/68; A61K 31/704

(52) U.S. Cl. 435/6; 536/37; 514/36

(57) ABSTRACT

The invention provides screening methods for the identification of compounds that inhibit nonstop degradation of mRNA, including compounds that inhibit the exosome. The invention further provides methods of treatment for genetic disorders caused by premature termination codons.

### RELATED APPLICATIONS

This application claims the benefit of U.S. Provisional Application Serial No. 60/373,093, filed Apr. 16, 2002, the entire contents of which is incorporated herein by this reference.

### GOVERNMENT SUPPORT

This work described herein was supported by a grant from National Institutes of Health (Grant No. GM55239). Therefore, the U.S. Government may have certain rights in the invention.

### BACKGROUND OF THE INVENTION

Approximately one third of inherited genetic disorders and various forms of cancer are caused by frameshift or nonsense mutations which result in the generation of a premature termination codon. Contrary to intuition, nonsense mutations frequently do not result in the production of truncated proteins. Rather, transcripts derived from nonsense containing alleles are specifically recognized and rapidly degraded by the nonsense-mediated mRNA decay (NMD) pathway. Recently, there have been many attempts to treat genetic disorders resulting from premature termination codons with long-term and high dose aminoglycoside regimens. However, there has been little clinical success using this these drugs, and the reason for their failure was not known. Additionally, aminoglycosides are toxic in high doses.

Accordingly, there currently exists a great need for treatments that can treat genetic diseases in general, and specifically, genetic diseases caused by premature termination codons.

Eukaryotes have evolved surveillance mechanisms that are intimately linked to translation to eliminate errors in mRNA biogenesis. The decay of transcripts containing premature termination codons (PTCs) by the nonsense mediated mRNA decay (NMD) pathway effectively prevents expression of deleterious truncated proteins. In prokaryotes, protein products encoded by transcripts lacking termination codons are marked for degradation by the addition of a COOH-terminal tag encoded by tmRNA (A. W. Karzai, E. D. Roche, R. T. Sauer, Nature Struct. Biol. 7, 449 (2000); K. C. Keiler, P. R. Waller, R. T. Sauer, Science 271, 990 (1996)). Thus, both the presence and context of translational termination can regulate gene expression.

### SUMMARY OF THE INVENTION

The present invention is based, at least in part, on the discovery of a novel pathway for the degradation of mRNA transcripts that do not contain any in-frame stop codons. The existence of this pathway, referred to alternately herein as “nonstop decay”, “nonstop degradation”, or “NSD”, is the reason that drugs used to promote readthrough of premature termination codons (PTCs) have not had clinical success. Accordingly, the present invention provides screening methods for the identification of compounds that can inhibit NSD. Such compounds, when used in conjunction with agents that promote readthrough of PTCs, are useful in the treatment of genetic diseases that are caused by PTCs. Accordingly, the present invention also provides methods of treatment.

In one embodiment, the present invention provides methods of identifying compounds capable of inhibiting nonstop degradation of mRNA comprising contacting a cell comprising a reporter gene lacking a termination codon with a test compound, measuring the level of expression or activity of the polypeptide encoded by the reporter gene, and comparing the level of expression or activity of the polypeptide encoded by the reporter gene to the level of expression or activity of the polypeptide encoded by the reporter gene in control cells, wherein a compound that upregulates the expression or activity of the polypeptide encoded by the reporter gene, as compared to the level of expression or activity of the polypeptide encoded by the reporter gene in control cells, is identified as a compound capable of inhibiting nonstop degradation of mRNA.

In another embodiment, the invention provides methods of identifying compounds capable of inhibiting nonstop degradation of mRNA comprising contacting a cell comprising a reporter gene having a premature stop termination codon with a test compound, contacting the cell with an aminoglycoside antibiotic, measuring the level of expression or activity of the polypeptide encoded by the reporter gene, and comparing the level of expression or activity of the polypeptide encoded by the reporter gene to the level of expression or activity of the polypeptide encoded by the reporter gene in control cells, wherein a compound that upregulates the expression or activity of the polypeptide encoded by the reporter gene, as compared to the level of expression or activity of the polypeptide encoded by the reporter gene in control cells, is identified as a compound capable of inhibiting nonstop degradation of mRNA. In a further embodiment, the aminoglycoside antibiotic is selected from the group consisting of G-418, gentamycin (also referred to as gentamicin), kanamycin, neomycin, netilmicin, paromomycin, streptomycin, tobramycin, hygromycin, amikacin, apramycin, and dihydrostreptomycin.

In a preferred embodiment, the reporter gene is contained within an expression vector. In other embodiments, the reporter gene encodes luciferase (e.g., firefly luciferase or Renilla luciferase), β-galactosidase, chloramphenicol acetyl transferase, or a fluorescent protein (e.g., green fluorescent protein, enhanced green fluorescent protein, red fluorescent protein, yellow fluorescent protein, enhanced yellow fluorescent protein, blue fluorescent protein, or cyan fluorescent protein).

In another embodiment, the cell used in the methods of the invention is a eukaryotic cell (e.g., a yeast cell or a mammalian cell, including a human cell).

In one embodiment, the level of expression of the polypeptide encoded by the reporter gene is measured by Western blotting, ELISA, or RIA. In another embodiment, the level of expression or activity of the polypeptide encoded by the reporter gene is determined by measuring luciferase activity (e.g., using a standard luciferase assay), β-galactosidase activity (e.g., using a standard P-galactosidase assay), or chloramphenicol acetyl transferase activity (e.g., using a standard chloramphenicol acetyl transferase assay), or by measuring the level of fluorescence of the fluorescent protein.

In another embodiment, the methods of the invention further comprise contacting a cell comprising a reporter gene lacking a termination codon with a test compound identified by any of the methods of described herein, measuring the half life of the reporter gene mRNA, comparing the half life of the reporter gene mRNA to the half life of the reporter gene mRNA in control cells, wherein a compound that increases the half life of the reporter gene mRNA, as compared to the half life of the reporter gene mRNA in control cells, is confirmed as a compound capable of inhibiting nonstop degradation of mRNA.

In still another embodiment, the methods of the invention further comprise contacting a cell comprising a reporter gene having a premature stop termination codon with a test compound identified by any of the methods described herein, contacting the cell with an aminoglycoside antibiotic, measuring the half life of the reporter gene mRNA, and comparing the half life of the reporter gene mRNA to the half life of the reporter gene mRNA in control cells, wherein a compound that increases the half life of the reporter gene mRNA, as compared to the half life of the reporter gene mRNA in control cells, is confirmed as a compound capable of inhibiting nonstop degradation of mRNA.

In a preferred embodiment, the level of expression of the reporter gene mRNA is measured by Northern blotting, primer extension, nuclease protection, or RT-PCR.

In still another embodiment, the invention provides methods of treating a genetic disorder in a subject caused by a premature termination codon comprising administering to the subject a therapeutically effective amount of an aminoglycoside antibiotic and a therapeutically effective amount of a compound that inhibits nonstop degradation of mRNA, thereby treating the genetic disorder in the subject. In a preferred embodiment, the disorder is muscular dystrophy (e.g., Duchenne muscular dystrophy or limb-girdle muscular dystrophy. In another preferred embodiment, the disorder is cystic fibrosis.

Other features and advantages of the invention will be apparent from the following detailed description and claims.

### BRIEF DESCRIPTION OF THE DRAWINGS

FIG. 1 depicts the results of a computer search performed using the human mRNA database and the S. cerevisiae ORF database and an analysis program written in PERL. 239 human mRNAs contain a polyadenylation signal consisting of either of the two most common words for the positioning, cleavage, and downstream elements (J. H. Graber, C. R. Cantor, S.C. Mohr, T. F. Smith, Proc. Natl. Acad. Sci. U.S.A. 96, 14055 (1999)), separated by optimal distances (30. F. Chen, C. C. MacDonald, J. Wilusz, Nucleic Acids Res. 23, 2614 (1995)), where the cleavage site occurs between the start and stop codons as determined from the cDNA coding sequence (CDS). Similarly 52 S. cerevisiae ORFs contained the yeast polyadenylation signal consisting of optimal upstream, positioning, and cleavage signals followed by any of nine common “U-rich” signals (24. J. H. Graber, C. R. Cantor, S. C. Mohr, T. F. Smith, Nucleic Acids Res. 27, 888 (1999)) with optimal spacing of the elements.

FIG. 2 depicts graphs of the half-lives of WT-PGK1 and Ter-poly(A)-PGK1 transcripts in wild-type-(WT) and SK17-deleted (ski7A) yeast strains treated with the indicated dose of paromomycin (mg/ml) (PM) for 20 hours. For cycloheximide (CHX) experiments, yeast were treated identically except 100 μg/ml of CHX was added during the last hour before half-life determination.

FIG. 3 depicts the results of a readthrough assay using a dual luciferase reporter construct.

### DETAILED DESCRIPTION OF THE INVENTION

The present invention is based, at least in part, on the discovery of a novel pathway for the degradation of mRNA transcripts that do not contain any in-frame stop codons. The existence of this pathway, referred to alternately herein as “nonstop decay”, “nonstop degradation”, or “NSD”, is the reason that drugs used to promote readthrough of premature termination codons (PTCs) have not had clinical success. Accordingly, the present invention provides screening methods for the identification of compounds that can inhibit NSD. Such compounds, when used in conjunction with agents that promote readthrough of PTCs, are useful in the treatment of genetic diseases that are caused by PTCs. Accordingly, the present invention also provides methods of treatment.

As used herein, a “genetic disease” or “genetic disorder” is a disease, disorder, syndrome, or condition that is caused by a mutation in a subject's germline DNA. Genetic diseases may also be caused, in some cases, by a mutation in a subjects somatic DNA. In a preferred embodiment, a genetic disease that may be treated using the methods of the invention is caused by a PTC in a particular gene. PTCs are most commonly caused by point mutations, but may also be caused by other types of mutations, included frameshifts or deletions that result in downstream, in-frame stop codons. Any disorder that is caused by a PTC may be treated by the methods of the invention. Exemplary disorders include, but are not limited to, cystic fibrosis (CF), Duchenne muscular dystrophy (DMD), Limb-Girdle Muscular Dystrophy (LGMD), familial adenomatous polyposis of the colon (FAPC), ataxia telangectasia, hemophilia A, hemophilia B, beta-thalassemia, androgen insensitivity syndrome, familial hypercholesterolemia, neurofibromatosis type 1, polycystic kidney disease, cholesteryl ester storage disease (CESD), Wolman disease, Charcot-Marie-Tooth syndrome, Alport syndrome, Hurler's syndrome, severe combined immune deficiency disorder (SCID), cancer (e.g., colon cancer, breast cancer, lung cancer, leukemias, lymphomas, and brain cancer), and congenital obesity due to a leptin receptor mutation.

As used interchangeably herein, the terms “stop codon” and “termination codon” refer nucleotide codons that do not code for any amino acid residues. Such codons, consisting of three nucleotides, when read by a translating ribosome, signal the ribosome to cease translation of the polypeptide. As used interchangeably herein, the terms “premature stop codon”, “premature termination codon”, and “PTC” refer to stop codons that occur abnormally in an mRNA, usually upstream of the normal stop codon. PTCs may result in the translation of a shortened polypeptide, or in degradation of the mRNA, as described elsewhere herein.

As used herein, the term “compound that induces readthrough of PTCs” includes any compound that, when applied to and/or present in a cell, induces ribosomes to read a stop codon, e.g., a PTC, as coding for an amino acid.

I. Screening Assays

In one embodiment, the invention provides methods (also referred to herein as “screening assays”) for identifying inhibitors, i.e., candidate or test compounds or agents (e.g., nucleic acids, peptides, peptidomimetics, small molecules, or other drugs) which inhibit the NSD pathway. In particular, such compounds are predicted by inhibit exosome activity, and may specifically target the exosome proteins and/or exosome associated proteins (e.g., the ski proteins).

In one embodiment, the invention provides assays for screening candidate or test compounds which are inhibitors of NSD. In another embodiment, the invention provides assays for screening candidate or test compounds which bind to or modulate the activity of the exosome or exosome associated proteins. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:45).

Examples of methods for the synthesis of molecular libraries can be found in the art, for example, in: DeWitt et al. (1993) Proc. Natl. Acad. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.).

In a preferred embodiment, an assay is a cell-based assay in which a cell which expresses a reporter gene that lacks a stop codon is contacted with a test compound and the ability of the test compound to induce expression and/or activity of the reporter protein is determined. In control cells, e.g., cells not contacted with the test compound, the reporter will not be expressed, or will be expressed at very low levels, because the NSD pathway will induce degradation of the reporter mRNA. However, if contacted with a test compound that inhibits the exosome, NSD will be inhibited, the reporter mRNA will not be degraded, and the reporter protein will be translated. As described elsewhere herein, the reporter can be any detectable marker. The reporter is a nucleic acid sequence that encodes a polypeptide, the expression of which can be measured by, for example, Western blotting, ELISA, or RIA assays. Reporter expression can also be monitored by measuring the activity of the polypeptide encoded by the reporter using, for example, a standard glutamate transport assay, a luciferase assay, a β-galactosidase assay, a chloramphenicol acetyl transferase (CAT) assay, or a fluorescent protein assay.

In another embodiment, the assay is a cell-based assay in which a cell which expresses a reporter gene that has a premature termination codon (PTC) is contacted with a test compound as well as a compound that induces readthrough of premature termination codons, and the ability of the test compound to induce expression and/or activity of the reporter protein is determined. In control cells, e.g., cells not contacted with the test compound, the compound that induces readthrough of PTCs allows readthrough of the PTC; however, the reporter will still not be expressed, or will be expressed at very low levels, because the NSD pathway will induce degradation of the reporter mRNA. However, if contacted with a test compound that inhibits the exosome, NSD will be inhibited, the reporter mRNA will not be degraded, and the reporter protein will be translated.

In a preferred embodiment, the compound that induces readthrough of PTCs is an aminoglycoside antibiotic (e.g., G-418, gentamycin, kanamycin, neomycin, netilmicin, paromomycin, streptomycin, tobramycin, hygromycin, amikacin, apramycin, and dihydrostreptomycin).

The cell used in the methods of the invention is preferably a eukaryotic cell (e.g., a yeast cell or a mammalian cell, including a human cell).

Compounds identified using the methods described above may be confirmed as inhibitors of NSD by performing cell-based assays similar to those above, and measuring the half-life of the reporter mRNA. Compounds that inhibit NSD preferably increase the half-life of reporter mRNAs. mRNA half-lives can be measured by any method known in the art, including, but not limited to, Northern blotting, primer extension, nuclease protection, or RT-PCR.

The ability of the test compound to bind to exosome proteins and/or exosome associated proteins can also be determined. Determining the ability of the test compound to bind to and/or modulate exosome proteins and/or exosome associated proteins can be accomplished, for example, by coupling the test compound, exosome proteins and/or exosome associated proteins with a radioisotope or enzymatic label such that binding of the exosome proteins and/or exosome associated proteins to the test compound can be determined by detecting the labeled component in a complex. For example, compounds (e.g., the test compound, exosome proteins and/or exosome associated proteins) can be labeled with ³²P, ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a test compound, exosome proteins and/or exosome associated proteins) to interact with the exosome proteins and/or exosome associated proteins without the labeling of any of the interactants. For example, a microphysiometer can be used to detect the interaction of a compound with exosome proteins and/or exosome associated proteins without the labeling of either the compound or the exosome proteins and/or exosome associated proteins (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and the Nonstop reporter gene.

In another embodiment, the assay is a cell-free assay in which exosome protein and/or exosome associated protein or portion thereof is contacted with a test compound and the ability of the test compound to modulate (e.g., stimulate or inhibit) the activity of the exosome protein and/or exosome associated protein is determined. Determining the ability of the test compound to modulate the activity of exosome proteins and/or exosome associated proteins can be accomplished, for example, by determining the ability of the exosome proteins and/or exosome associated proteins to bind to exosome proteins and/or exosome associated proteins target molecules by one of the methods described above for determining direct binding. Determining the ability of the exosome proteins and/or exosome associated proteins to bind to exosome proteins and/or exosome associated proteins target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In yet another embodiment, the cell-free assay involves contacting exosome proteins and/or exosome associated proteins or portion thereof with a known compound which binds the exosome proteins and/or exosome associated proteins to form an assay mixture, contacting the assay mixture with a test compound, and determining the ability of the test compound to interact with the exosome proteins and/or exosome associated proteins, wherein determining the ability of the test compound to interact with the exosome proteins and/or exosome associated proteins comprises determining the ability of the exosome proteins and/or exosome associated proteins to preferentially bind to or modulate the activity of an exosome protein and/or exosome associated protein target molecule.

In more than one embodiment of the above assay methods of the present invention, it may be desirable to immobilize either exosome proteins and/or exosome associated proteins or target molecule to facilitate separation of complexed from uncomplexed forms of one or both of the molecules, as well as to accommodate automation of the assay. Binding of a test compound to exosome proteins and/or exosome associated proteins e, or interaction of exosome proteins and/or exosome associated proteins with a substrate or target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/target fusion proteins can be adsorbed onto glutathione sepharose beads (Sigma Chemical, St. Louis, Mo.) or glutathione derivatized micrometer plates, which are then combined with the test compound or the test compound and either the non-adsorbed target protein or exosome proteins and/or exosome associated proteins, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of exosome protein and/or exosome associated protein binding or activity determined using standard techniques.

Other techniques for immobilizing proteins or nucleic acids on matrices can also be used in the screening assays of the invention. For example, either exosome proteins and/or exosome associated proteins or target molecule can be immobilized utilizing conjugation of biotin and streptavidin. Biotinylated exosome proteins and/or exosome associated proteins, substrates, or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with exosome proteins and/or exosome associated proteins or target molecules but which do not interfere with binding of the exosome protein and/or exosome associated protein to its target molecule can be derivatized to the wells of the plate, and unbound target or exosome proteins and/or exosome associated proteins trapped in the wells by antibody conjugation. Methods for detecting such complexes, in addition to those described above for the GST-immobilized complexes, include immunodetection of complexes using antibodies reactive with the exosome proteins and/or exosome associated proteins or target molecule, as well as enzyme-linked assays which rely on detecting an enzymatic activity associated with the exosome proteins and/or exosome associated proteins or target molecule.

In yet another aspect of the invention, the exosome proteins and/or exosome associated proteins can be used as “bait” in a one-hybrid assay (see, e.g., BD Matchmaker One-Hybrid System (1995) Clontechniques X(3):2-4; BD Matchmaker Library Construction & Screening Kit (2000) Clontechniques XV(4):5-7; BD SMART technology overview (2002) Clontechniques XVII(1):22-28; Ausubel, F. M., et al. (1998 et seq.) Current Protocols in Molecular Biology Eds. Ausubel, F. M., et al., pp. 13.4.1-13.4.10) to identify proteins which bind to or interact with the exosome proteins and/or exosome associated proteins (“exosome proteins and/or exosome associated proteins-binding proteins” or “exosome proteins and/or exosome associated proteins gene-bp”) and are involved in exosome activity. Such exosome proteins and/or exosome associated proteins—binding proteins are also likely to be involved in the regulation of NSD.

In another aspect, the invention pertains to a combination of two or more of the assays described herein. For example, a modulating agent can be identified using a cell-based or a cell-free assay, and the ability of the agent to modulate the activity of NSD can be confirmed in vivo, e.g., in an animal such as an animal model for a genetic disease.

This invention further pertains to novel agents identified by the above-described screening assays. Accordingly, it is within the scope of this invention to further use an agent identified as described herein in an appropriate animal model (e.g., an animal model for a genetic disease). For example, an agent identified as described herein (e.g., an NSD modulating agent or an exosome binding protein) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

II. Recombinant Expression Vectors and Host Cells

Another aspect of the invention pertains to vectors, for example recombinant expression vectors, containing a reporter gene. As used herein, the term ‘vector’ refers to a nucleic acid molecule capable of transporting another nucleic acid to which it has been linked. One type of vector is a ‘plasmid’, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as ‘expression vectors’. In general, expression vectors of utility in recombinant DNA techniques are often in the form of plasmids. In the present specification, ‘plasmid’ and ‘vector’ can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, ‘operably linked’ is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term ‘regulatory sequence’ is intended to include promoters, enhancers and other expression control elements (e.g. polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). In a preferred embodiment, the regulatory sequences in the expression vectors of the invention are derived from the Nonstop reporter gene of the invention, and the nucleic acid sequence to be expressed is a reporter gene, as described elsewhere herein. It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

As used herein a “reporter” or a “reporter gene” refers to a nucleic acid molecule encoding a detectable marker. Preferred reporter genes include luciferase (e.g., firefly luciferase or Renilla luciferase), β-galactosidase, chloramphenicol acetyl transferase (CAT), and a fluorescent protein (e.g., green fluorescent protein, red fluorescent protein, yellow fluorescent protein, blue fluorescent protein, cyan fluorescent protein, or variants thereof, including enhanced variants). Reporter genes must be detectable by a reporter assay. Reporter assays can measure the level of reporter gene expression or activity by any number of means, including measuring the level of reporter mRNA, the level of reporter protein, or the amount of reporter protein activity.

In preferred embodiments of the invention, the reporter genes used in the methods (e.g., the screening assays) lack stop codons. Stop codons can be removed from any reporter gene using standard methods known in the art, including site-directed mutagenesis. In another preferred embodiment, the reporter genes used in the methods of the invention contain a premature termination codons. Premature termination codons can be inserted in any reporter gene using standard methods known in the art, including site-directed mutagenesis.

Methods for measuring mRNA levels are well-known in the art and include, but are not limited to, Northern blotting, RT-PCR, primer extension, and nuclease protection assays. Methods for measuring reporter protein levels are also well-known in the art and include, but are not limited to, Western blotting, ELISA, and RIA assays. Reporter activity assays are still further well-known in the art, and include luciferase assays, β-galactosidase, and chloramphenicol acetyl transferase (CAT) assays. Fluorescent protein activity can be measured by detecting fluorescence.

The recombinant expression vectors of the invention are preferably designed for expression in eukaryotic cells (e.g., mammalian cells). Alternatively, the recombinant expression vector can be transcribed and translated in vitro.

Another aspect of the invention pertains to host cells into which a reporter gene nucleic acid molecule of the invention is introduced, e.g., an reporter gene nucleic acid molecule within a vector (e.g., a recombinant expression vector) or a reporter gene nucleic acid molecule containing sequences which allow it to homologously recombine into a specific site of the host cell's genome. The terms ‘host cell’ and ‘recombinant host cell’ are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a vector containing a reporter gene can be propagated and/or expressed in bacterial cells such as E. coli, insect cells, yeast or mammalian cells (such as Chinese hamster ovary cells (CHO), COS cells (e.g., COS7 cells), C6 glioma cells, HEK 293T cells, or neurons). Other suitable host cells are known to those skilled in the art.

Vector DNA can be introduced into prokaryotic or eukaryotic cells via conventional transformation or transfection techniques. As used herein, the terms ‘transformation’ and ‘transfection’ are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify an d select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a reporter gene or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an mRNA or protein (e.g., a reporter mRNA or protein) encoded by the nucleic acid molecule operatively linked to the reporter gene. Accordingly, the invention further provides methods for producing an mRNA or protein using the host cells of the invention. In one embodiment, the method comprises culturing the host cell of the invention (into which a recombinant expression vector containing the reporter gene has been introduced) in a suitable medium such that mRNA and/or protein encoded by the operatively linked nucleic acid molecule is produced. In another embodiment, the method further comprises isolating the mRNA and/or protein from the medium or the host cell.

The host cells of the invention can also be used to produce non-human transgenic animals. For example, in one embodiment, a host cell of the invention is a fertilized oocyte or an embryonic stem cell into which Nonstop reporter gene sequences have been introduced. Such host cells can then be used to create non-human transgenic animals in which exogenous reporter gene sequences have been introduced into their genome or homologous recombinant animals in which endogenous reporter gene sequences have been altered. Such animals are useful for studying the function and/or activity of compounds which can inhibit NSD. As used herein, a ‘transgenic animal’ is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal.

A transgenic animal of the invention can be created by introducing a reporter gene-encoding nucleic acid into the male pronuclei of a fertilized oocyte, e.g., by microinjection or retroviral infection, and allowing the oocyte to develop in a pseudopregnant female foster animal. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of a reporter gene transgene in its genome and/or expression of a reporter gene operatively linked to the reporter gene transgene in tissues or cells of the animals. A transgenic founder animal can then be used to breed additional animals carrying the transgene. Moreover, transgenic animals carrying a transgene containing an Nonstop reporter gene can further be bred to other transgenic animals carrying other transgenes.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

As used herein, the term “standard assay”, e.g., “standard luciferase assay”, “standard β-galactosidase assay”, and “standard chloramphenicol acetyltransferase assay” refer to standard methods known in the art for measuring the activity of luciferase, β-galactosidase, and chloramphenicol acetyltransferase. The fluorescence level of fluorescent proteins may be measured using standard methods known in the art.

Unless specifically indicated otherwise, all of the embodiments of the invention use standard molecular biology and biochemical methods to produce. A wide variety of molecular and biochemical methods are available for generating and expressing the vectors and constructs of the present invention; see e.g. the procedures disclosed in Molecular Cloning, A Laboratory Manual (2nd Ed., Sambrook, Fritsch and Maniatis, Cold Spring Harbor), Current Protocols in Molecular Biology (Eds. Ausubel, Brent, Kingston, More, Freidman, Smith and Struhl, Greene Publ. Assoc., Wiley-Interscience, NY, N.Y. 1992) or other procedures that are otherwise known in the art.

III. Methods of Treatment

In one embodiment, the present invention provides methods of treating genetic disorders which comprise administering a therapeutically effective amount of a pharmaceutical composition comprising a compound that induces readthrough of PTCs and a therapeutically effective amount of a compound that inhibits NSD to a subject (e.g., a mammal such as a human).

As described elsewhere herein, compounds that induce readthrough of PTCs are useful for the treatment of genetic diseases caused by PTCs because they allow the translation past the mutant stop codon. However, because these compounds allow all readthrough, they result in mRNAs that effectively have no stop codons, resulting in induction of the NSD pathway, degradation of the mRNA, and no translated protein. Therefore, administration of compounds identified using the methods described herein as inhibitors of NSD will prevent degradation of the mRNAs, and will allow compounds that induce readthrough of PTCs to have clinical effectiveness in the treatment of genetic disorders.

In a preferred embodiment, a compound that induce readthrough of PTCs is an aminoglycoside antibiotic (e.g., G-418, gentamycin, kanamycin, neomycin, netilmicin, paromomycin, streptomycin, tobramycin, hygromycin, amikacin, apramycin, or dihydrostreptomycin).

Compounds that inhibit NSD may be identified using the methods described herein. siRNAs (small interfering RNAs) that inhibit expression of the exosome proteins or exosome associated proteins.

The preferred therapeutic methods of the invention (which include prophylactic treatment) in general comprise administration of a therapeutically effective amount of a pharmaceutical composition comprising a compound that induces readthrough of PTCs and a therapeutically effective amount of a compound that inhibits NSD to an animal in need thereof, including a mammal, particularly a human. The compounds may be provided in the same pharmaceutical composition, or as separate compositions. Such treatment will be suitably administered to subjects, particularly humans, suffering from, having, susceptible to, or at risk for a genetic disorder.

As will be apparent to those of skill in the art, a subject will only benefit from the treatment methods of the invention if the subject's genetic disorder is caused by a PTC. Accordingly, subjects should preferably be tested prior to commencement of treatment to determine whether the gene that causes the disease contains a PTC. However, not all subjects having a disease-causing PTC will benefit from the treatment methods described herein. For example, if a PTC occurs at a site of a critical amino acid residue, the insertion of a different amino acid in place of the PTC may cause a non-function protein. Additionally, some PTCs are caused by frameshifts or deletions that are still present even after readthrough of the PTC. Accordingly, subjects will have to be tested and monitored individually to determine whether they will benefit from the treatment methods of the invention.

For therapeutic applications, compounds of the invention may be suitably administered to a subject such as a mammal, particularly a human, alone or as part of a pharmaceutical composition, comprising the compound together with one or more acceptable carriers thereof and optionally other therapeutic ingredients. The carrier(s) must be “acceptable” in the sense of being compatible with the other ingredients of the formulation and not deleterious to the recipient thereof.

The pharmaceutical compositions of the invention include those suitable for oral, rectal, nasal, topical (including buccal and sublingual), vaginal or parenteral (including subcutaneous, intramuscular, intravenous and intradermal) administration. The formulations may conveniently be presented in unit dosage form, e.g., tablets and sustained release capsules, and in liposomes, and may be prepared by any methods well know in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier which constitutes one or more accessory ingredients. In general, the compositions are prepared by uniformly and intimately bringing into association the active ingredients with liquid carriers, liposomes or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Application of the subject therapeutics often will be local, so as to be administered at the site of interest. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

It will be appreciated that actual preferred amounts of a compound of the invention used in a given therapy will vary to the particular active compound being utilized, the particular compositions formulated, the mode of application, the particular site of administration, the patient's weight, general health, sex, etc., the particular indication being treated, etc. and other such factors that are recognized by those skilled in the art including the attendant physician or veterinarian. Optimal administration rates for a given protocol of administration can be readily determined by those skilled in the art using conventional dosage determination tests.

As used herein, “treatment” of a subject includes the application or administration of a therapeutic agent to a subject (e.g., the compounds of the present invention), or application or administration of a therapeutic agent to a cell or tissue from a subject, who has a disease (e.g., a genetic disorder) or disorder, has a symptom of a disease or disorder, or is at risk of (or susceptible to) a disease or disorder, with the purpose of curing, healing, alleviating, relieving, altering, remedying, ameliorating, improving, or affecting the disease or disorder, the symptom of the disease or disorder, or the risk of (or susceptibility to) the disease or disorder.

This invention is further illustrated by the following examples which should not be construed as limiting. The contents of all references, patents, and published patent applications cited throughout this application, as well as the figures and the Sequence Listing, are incorporated herein in their entirety by reference.

### EXAMPLES

### EXAMPLE 1

### NSD: An mRNA Surveillance Mechanism that Eliminates Transcripts Lacking Termination Codons

In the following Example, numbers within parentheses refer to the notes and references found at the end of the Example.

In order to determine whether the presence of translational termination influences mRNA stability, we assayed PGK1 tran-scripts in Saccharomyces cerevisiae derived from the following constructs (3): wild-type PGK1 (WT-PGK1), a nonsense form of PGK1 harboring a PTC at codon 22 [PTC(22)-PGK1], and nonstop-PGK1 that was created by removing the bona fide termination codon and all in-frame termination codons in the 3′ UTR (untranslated region) from the WT-PGK1 transcript. Nonstop-PGK1 transcripts were as labile as their nonsense-containing counterparts. At least three trans-acting factors (Upf1p, Upf2p, and Upf3p) are essential for nonsense-mediated mRNA decay (NMD) in S. cerevisiae (4, 5). Remarkably, nonstop transcripts were not stabilized in strains lacking Upf1p, distinguishing the pathway of decay from NMD.

The turnover of normal mRNAs requires deadenylation followed by Dcp1p-mediated decapping and degradation by the major 5′-to-3′ exonuclease Xrn1p (6, 7). NMD is distinguished in that these events occur without prior deadenylation (8, 9). Nonstop-PGK1 transcripts showed rapid decay in strains lacking Xrn1p or Dcp1p activity, providing further evidence that they are not subject to NMD. This result was surprising since both of these factors are also required for the turnover of normal mRNAs after deadenylation by the major deadenylase Ccr4p (10). Nonstop decay was also unaltered in a strain lacking Ccr4p. Therefore, degradation of nonstop-PGK1 transcripts requires none of the factors involved in the pathway for degradation of wild-type or nonsense mRNAs.

Additional experiments were performed to assess the role of translation in nonstop decay. Treatment with cycloheximide (CHX) or depletion of charged tRNAs in yeast harboring the conditional cca1-1 allele (11) grown at the nonpermissive temperature substantially increased the stability of nonstop-PGK1 transcripts. It has been shown that translation into the 3′ UTR of selected transcripts can displace bound trans-factors that are positive determinants of message stability (13, 14). To determine whether this mechanism is relevant to nonstop decay, we examined the performance of transcript Ter-poly(A)-PGK1, which contains a termination codon inserted one codon upstream of the site of polyadenylate [poly(A)] addition (15) in transcript nonstop-PGK1. The addition of a termination codon at the 3′ end of the 3′ UTR of a nonstop transcript resulted in substantial (threefold) stabilization. Unlike nonstop-PGK1 transcripts, the half-life of Ter-poly(A)-PGK1 was increased in the absence of Xrn1p, indicating that this transcript is degraded by the pathway for normal mRNA and not by the nonstop pathway. These data suggest that the instability of nonstop-PGK1 transcripts cannot fully be explained by ribosomal displacement of factors bound to the 3′ UTR.

There is an emerging view that the 5′ and 3′ ends of mRNAs interact to form a closed loop and that this conformation is required for efficient translation initiation and normal mRNA stability. Participants in the interaction include components of the translation initiation complex eIF4F as well as Pab1p. The absence of a termination codon in nonstop-PGK1 is predicted to allow the ribosome to continue translating through the 3′ UTR and poly(A) tail, potentially resulting in displacement of Pab1p, disruption of normal mRNP structure, and consequently accelerated degradation of the transcript. However, while mRNAs in pab1Δ strains do undergo accelerated decay, the rapid turnover is a result of premature decapping and can be suppressed by mutations in XRN1 (16), allowing distinction from nonstop decay. Lability of nonstop transcripts might also be a consequence of ribosomal stalling at the 3′ end of the transcript. It may be that 3′-to-5′ exonucleolytic activity underlies the decapping- and 5′-to-3′ exonuclease-independent, translation-dependent accelerated decay of nonstop transcripts. An appealing candidate is the exosome, a collection of proteins with 3′-to-5′ exoribonuclease activity that functions in the processing of 5.8S RNA, rRNA, small nucleolar RNA (snoRNA), small nuclear RNA (snRNA), and other transcripts. Data presented below in Example 2 validate this prediction.

In order to determine whether nonstop decay functions in mammals, we assessed the performance of transcripts derived from β-glucuronidase (βgluc) minigene constructs containing exons 1, 10, 11, and 12 separated by introns derived from the endogenous gene (18). The abundance of transcripts derived from a nonstop-βgluc version of this minigene was significantly reduced relative to WT-βgluc, suggesting that a termination codon is also essential for normal mRNA stability in mammalian cells. Addition of a stop codon one codon upstream from the site of poly(A) addition in nonstop-βgluc [Ter-poly(A)-βgluc (15)] increased the abundance of this transcript to near-wildtype levels. Thus, all functional characteristics of nonstop transcripts in yeast appear to be relevant to mammalian systems.

Both the abundance and stability of most nonsense transcripts, including nonsense-βgluc, are reduced in the nuclear fraction of mammalian cells (19, 20). This has been interpreted to suggest that translation and NMD initiate while the mRNA is still associated with (if not within) the nuclear compartment. If translation is initiated on nucleus-associated transcripts, so might nonstop decay. Subcellular fractionation studies localized mammalian nonstop decay to the cytoplasmic compartment, providing further distinction from NMD.

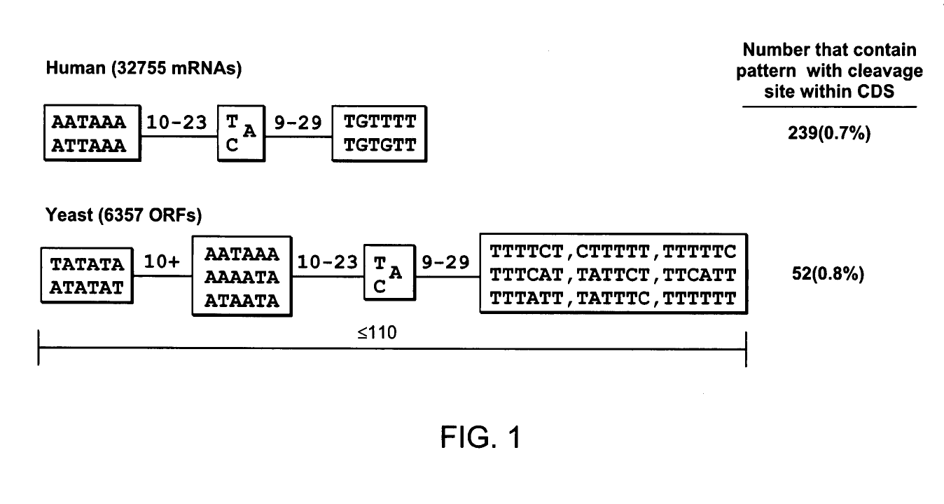

The conservation of nonstop decay in yeast and mammals suggests that the pathway serves an important biologic role. There are many potential physiologic sources of nonstop transcripts that warrant consideration. Mutations in bona fide termination codons would not routinely initiate nonstop decay due to the frequent occurrence of in-frame termination codons in the 3′ UTR and could not plausibly provide the evolutionary pressure for maintenance of nonstop decay. In contrast, alternative use of 3′-end processing signals embedded in coding sequence has been documented in many genes including CBP1 in yeast and the growth hormone receptor (GHR) gene in fowl (21-23). Moreover, a computer search of the human mRNA and S. cerevisiae open reading frame (ORF) databases revealed many additional genes that contain a strict consensus sequence for 3′-end cleavage and polyadenylation within their coding region (FIG. 1). Utilization of these premature signals would direct formation of truncated transcripts that might be substrates for the nonstop decay pathway. Indeed, analysis of 3425 random yeast cDNA clones sequenced from the 3′ end (i.e., 3′ ESTs) revealed that 40 showed apparent premature polyadenylation within the coding region (24).

The truncated GHR transcript in fowl is apparently translated, as evidenced by its association with polysomes (22). As predicted for a substrate for nonstop decay, the ratio of truncated-to-full-length GHR transcripts was dramatically increased after treatment of cultured chicken hepatocellular carcinoma cells with the translational inhibitor emetine. Other truncated transcripts, both bigger and smaller than the predicted 0.7-kb nonstop transcript, did not show a dramatic increase in steady-state abundance upon translational arrest suggesting that they are derived from other mRNA processing events, perhaps alternative splicing. To directly test whether the nonstop mRNA pathway degrades prematurely polyadenylated mRNAs, we analyzed CBP1 transcripts in yeast. The CBP1 gene produces a 2.2-kb full-length mRNA and a 1.2-kb species with premature 3′ end processing and polyadenylation within the coding region (25). As expected from the observation that nonstop decay requires the exosome in yeast (See Example 2), deletion of the gene encoding the exosome component Ski7p stabilized the prematurely polyadenylated mRNA but had no effect on the stability of the full-length mRNA. These data indicate that physiologic transcripts arising from premature polyadenylation are subject to nonstop mRNA decay. The cytoplasmic localization of ski7p is consistent with our observation that nonstop decay occurs within the cytoplasm.

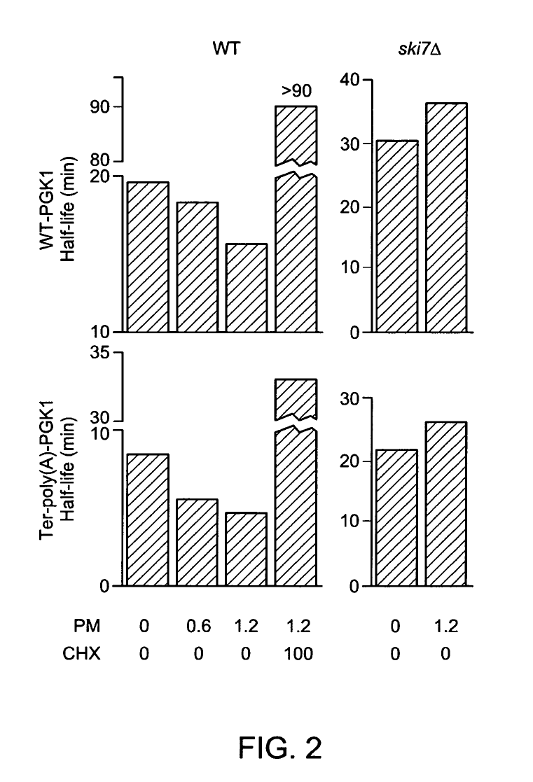

Any event that diminishes translational fidelity and promotes readthrough of termination codons could plausibly result in the generation of substrates for nonstop decay. In view of recent attempts to treat genetic disorders resulting from PTCs with long-term and high-dose aminoglycoside regimens, this may achieve medical significance. As a proof-of-concept experiment, we examined the performance of transcripts with one [Ter-poly(A)-PGK1] or multiple (WT-PGK1) in-frame termination codons (including those in the 3′ UTR) in yeast strains after treatment with paromomycin, which induces ribosomal readthrough. Remarkably, both transcripts showed a dose-dependent decrease in stability that could be reversed by inhibiting translational elongation with CHX or prevented by deletion of the gene encoding Ski7p (FIG. 2). These data suggest that nonstop decay can limit the efficiency of therapeutic strategies aimed at enhancing nonsense suppression and might contribute to the toxicity associated with aminoglycoside therapy.

The degradation of nonstop transcripts may be regulated. The relative expression level of truncated GHR transcripts compared to full-length transcripts varies in a tissue-, gender-, and developmental stage-specific manner (22) and the relative abundance of truncated CBP1 transcripts varies with growth condition (21). Data presented herein warrant the hypothesis that regulation may occur at the level of nonstop transcript stability rather than production. The conservation of the GHR coding sequence 3′-end processing signal throughout avian phylogeny and conservation of premature polyadenylation of the yeast RNA14 transcript and its fruitfly homolog su(f) support speculation that nonstop transcripts or derived protein products serve essential developmental and/or homeostatic functions that are regulated by nonstop decay (21, 26).

Many processes contribute to the precise control of gene expression including transcriptional and translational control mechanisms. In recent years, mRNA stability has emerged as a major determinant of both the magnitude and fidelity of gene expression. Perhaps the most striking and comprehensively studied example is the accelerated decay of transcripts harboring PTCs by the NMD pathway. Nonstop decay now serves as an additional example of the critical role that translation plays in monitoring the fidelity of gene expression, the stability of aberrant or a typical transcripts, and hence the abundance of truncated proteins.

### REFERENCES AND NOTES

Numbers contained within parentheses in Example 1 (above) refer to the following notes and references. All of the following notes and references are incorporated herein by reference.

1. A. W. Karzai, E. D. Roche, R. T. Sauer, Nature Struct. Biol. 7, 449 (2000).

2. K. C. Keiler, P. R. Waller, R. T. Sauer, Science 271, 990 (1996).

3. The nonstop-PGK1 construct was generated from WT-PGK1 [pRP469 (D. Muhlrad, R. Parker, Nature 370, 578 (1994))] by creating three point mutations, using site-directed mutagenesis (Quik-Change Site-Directed Mutagenesis Kit, Stratagene, La Jolla, Calif.) which eliminated the bona fide termination codon and all in-frame termination codons in the 3′ UTR. The Ter-poly(A)-PGK1 construct was created from the nonstop-PGK1 construct, using site-directed mutagenesis (Quik-Change Site-Directed Mutagenesis Kit, Stratagene, La Jolla, Calif.). All changes were confirmed by sequencing. Primer sequences are available upon request. The PTC(22)-PGK1 construct is described in (D. Muhlrad, R. Parker, Nature 370, 578 (1994)) (pRP609).

4. P. Leeds, J. M. Wood, B. S. Lee, M. R. Culbertson, Mol. Cell. Biol. 12, 2165 (1992).

5. Y. Cui, K. W. Hagan, S. Zhang, S. W. Peltz, Genes Dev. 9, 423 (1995).

6. D. Muhlrad, C. J. Decker, R. Parker, Genes Dev. 8, 855 (1994).

7. C. A. Beelman et al., Nature 382, 642 (1996).

8. D. Muhlrad, R. Parker, Nature 370, 578 (1994).

9. G. Caponigro, R. Parker, Microbiol. Rev. 60, 233 (1996).

10. M. Tucker et al., Cell 104, 377 (2001).

11. C. L. Wolfe, A. K. Hopper, N. C. Martin, J. Biol. Chem. 271, 4679 (1996).

13. I. M. Weiss, S. A. Liebhaber, Mol. Cell. Biol. 14, 8123 (1994).

14. X. Wang, M. Kiledjian, I. M. Weiss, S. A. Liebhaber, Mol. Cell. Biol. 15, 1769 (1995).

15. We performed 3′ rapid amplification of cDNA ends (RACE) using the Marathon cDNA Amplification Kit (Clontech, Palo Alto, Calif.) and the Advantage 2 Polymerase Mix (Clontech, Palo Alto, Calif.) according to the manufacturer's instructions. Poly(A) RNA isolated from cells (Oligotex mRNA midi kit, Qiagen, Valencia, Calif.) transiently transfected with WT-βgluc or from a wild-type yeast strain carrying a plasmid expressing WT-PGK1 was used as template. A single RACE product was generated, cloned (TOPO TA 2.1 kit, Invitrogen, Carlsbad, Calif.), and sequenced.

16. G. Caponigro, R. Parker, Genes Dev. 9, 2421 (1995).

18. To create the WT-βgluc minigene construct, mouse genomic DNA was used as template for amplifying three different portions of the β-glucuronidase gene: from nucleotide 2313 to 3030 encompassing exon 1 and part of intron 1 (including the splice donor sequence), from nucleotide 11285 to 13555 encompassing part of intron 9 (including the splice acceptor sequence), exon 10, and part of intron 10 (including the splice donor sequence), and from 13556 to 15813 which included the remainder of intron 10, exon 11, intron 11, and exon 12. All three fragments were TA cloned (Topo TA 2.1 kit, Invitrogen, Carlsbad, Calif.), sequenced, and then cloned into pZeoSV2 (Invitrogen, Carlsbad, Calif.). A single base-pair deletion corresponding to the gus^{mps} allele was generated by site-directed mutagenesis (Quik-Change Site-Directed Mutagenesis Kit, Stratagene, La Jolla, Calif.) of the WT-βgluc construct to create nonsense-βgluc. Nonstop-βgluc was generated from WT-βgluc by two rounds of site-directed mutagenesis (Quik-Change Site-Directed Mutagenesis Kit, Stratagene, La Jolla, Calif.), which removed the bona fide termination codon and all in-frame termination codons in the 3′ UTR. Ter-poly(A)-βgluc was generated from nonstop-βgluc via a single point mutation, which created a stop one codon upstream of the poly(A) tail. All changes were confirmed by sequencing.

19. P. Belgrader, J. Cheng, X. Zhou, L. S. Stephenson, L. E. Maquat, Mol. Cell. Biol. 14, 8219 (1994).

20. O. Kessler, L. A. Chasin, Mol. Cell. Biol. 16, 4426 (1996).

21. K. A. Sparks, C. L. Dieckmann, Nucleic Acids Res. 26, 4676 (1998).

22. E. R. Oldham, B. Bingham, W. R. Baumbach, Mol. Endocrinol. 7, 1379 (1993).

23. C. Hilger, I. Velhagen, H. Zentgraf, C. H. Schroder, J. Virol. 65, 4284 (1991).

24. J. H. Graber, C. R. Cantor, S. C. Mohr, T. F. Smith, Nucleic Acids Res. 27, 888 (1999).

25. K. A. Sparks, S. A. Mayer, C. L. Dieckmann, Mol. Cell. Biol. 17, 4199 (1997).

26. A. Audibert, M. Simonelig, Proc. Natl. Acad. Sci. U.S.A. 95, 14302 (1998).

27. All PGK1 minigene constructs were under the control of the GAL1 upstream activation sequence (UAS). Cultures were grown to mid-log phase in synthetic complete media-uracil (SC-ura) containing 2% galactose (transcription on). Cells were pelleted and resuspended in SC-ura media. Glucose was then added (transcription off) to a final concentration of 2% and aliquots were removed at the indicated time points. Approximately 10 μg of total RNA isolated with the hot phenol method (P. Leeds, J. M. Wood, B. S. Lee, M. R. Culbertson, Mol. Cell. Biol. 12, 2165 (1992)) was electrophoresed on a 1.2% agarose formaldehyde gel, transferred to a nylon membrane (Gene-Screen Plus, NEN, Boston, Mass.), and hybridized with a 23 bp oligonucleotide end-labeled radioactive probe that specifically detects the polyG tract in the 3′ UTR of the PGK1 transcripts (D. Muhlrad, R. Parker, Nature 370, 578 (1994)). Half-lives were determined by plotting the percent of mRNA remaining versus time on a semi-log plot. All half-lives were determined at 25° C. except for the ccr4Δ (performed at 30° C.) and dcp1-2 [performed after shift to the nonpermissive temp (37° C.) for 1 hour] experiments.

28. HeLa cells [maintained in DMEM with 10% fetal bovine serum (FBS)] were grown to ˜80 to 90% confluency, trypsinized, and resuspended in 300 μl of RPMI 1640 without FBS, 10 mM glucose and 0.1 mM dithiothreitol (DTT). Plasmid DNA (10 μg) was added and cells were electroporated (BioRad, Gene Pulser II Electroporation System, BioRad, Hercules, Calif.) at a voltage of 0.300 kV and a capacitance of 500 μF. Poly(A) RNA (Oligotex midi kit, Qiagen, 2 μg), isolated 36 hours after transfection, was separated on a 1.6% agarose formaldehyde gel, transferred to a nylon membrane, and hybridized with a polymerase chain reaction (PCR) product comprising exons 10 through 12 of the β-gluc gene that had been radioactively labeled by nick translation (Random Primed DNA Labeling Kit, Boehringer Mannheim, Indianapolis, Ind.). The membrane was subsequently stripped with boiling 0.5% SDS and probed with radioactively labeled zeocin cDNA. Northern blot results were quantitated using an Instant Imager (Packard Bioscience, Boston, Mass.). For subcellular fractionation, transiently transfected HeLa cells were trypsinized, washed in cold 1× phosphate buffered saline, and pelleted by centrifuging at 2040 g for 5 min at 4° C. Cells were then resuspended in 200 μl of 140 mM NaCl, 1.5 mM MgCl₂, 10 mM Tris-HCl (pH 8.6), 0.5% NP-40, and 1 mM DTT, vortexed, and incubated on ice for 5 min. Nuclei were pelleted by centrifuging at 12,000 g for 45 s at 4° C. and subsequently separated from the aqueous (cytoplasmic) phase.

29. J. H. Graber, C. R. Cantor, S. C. Mohr, T. F. Smith, Proc. Natl. Acad. Sci. U.S.A. 96, 14055 (1999).

30. F. Chen, C. C. MacDonald, J. Wilusz, Nucleic Acids Res. 23, 2614 (1995).

31. Plasmid pG-26 (25) containing the CBP1 gene under the control of the GAL promoter and the LEU2 selectable marker was introduced into wild-type and SK17-deleted strains. Yeast were grown in media lacking leucine and containing 2% galactose at 30° C. to an OD 600 of 0.5. Cells were pelleted and resuspended in media lacking a carbon source. Glucose was then added to a final concentration of 2% at time 0 and time points were taken. RNA was extracted and analyzed by northern blotting with a labeled oligo probe oRP1067 (5′-CTCGGTCCTG-TACCGAACGAGACGAGG-3′ (SEQ ID NO: 1).

### EXAMPLE 2

### Exosome-Mediated Recognition and Degradation of mRNAS Lacking a Termination Codon

In the following Example, numbers within parentheses refer to the notes and references found at the end of the Example.

As shown above in Example 1, degradation of a PGK1 mRNA, from which all in-frame termination codons have been removed (nonstop-PGK1), requires none of the enzymes involved in the major pathway for mRNA degradation, which occurs by deadenylation, decapping, and 5′-to-3′ digestion (3-5). This suggests that nonstop mRNAs might be degraded by the exosome complex of 3′-to-5′ exoribonucleases, the functions of which include 3′-to-5′ degradation of mRNA in the cytoplasm, nuclear processing of ribosomal RNA and small nucleolar RNAs, and degradation of processing intermediates and stalled mRNAs in the nucleus (6-8).

To test whether the exosome functions in nonstop decay, we first examined non-stop decay in a ski4-1 strain of yeast. The ski4-1 allele encodes a point mutation in one of the core exosome subunits that specifically disrupts cytoplasmic 3′-to-5′ degradation of mRNA without affecting any of the other known functions of the exosome (9). The ski4-1 mutation stabilizes the nonstop-PGK1 mRNA at least six-fold. Exosome-mediated degradation of normal cellular mRNAs requires the exosome and two other factors (6, 9). One factor is a heterotrimeric helicase complex of Ski2p, Ski3p, and Ski8p (6, 10). Ski2, −3, and −8 were all required for nonstop mRNA degradation. The second factor required for exosome-mediated mRNA decay was Ski7p, and deletion of SKI7 also caused stabilization of nonstop mRNAs. Because Ski2p and Ski7p localize to the cytoplasm (10, 11), we interpret these observations to indicate that nonstop mRNAs are degraded 3′ to 5′ by the cytoplasmic exosome. (Methods: Nonstop-PGK1 mRNA stability was measured in wild-type, ski4-1, ski2Δ, ski3Δ, ski8Δ, ski7Δ, ski7-ΔC, and ski7-ΔN strains. Each strain contained a URA3 plasmid encoding the reporter gene and was grown to early- to mid-log phase at 30° C. in media containing 2% galactose and lacking uracil. Transcription of the reporter gene was inhibited by replacing the media with media containing glucose (T=0 min) and aliquots were taken thereafter. RNA was analyzed as described in (9). The half-lives are averages of at least two experiments and were calculated after correction for loading (9).)

Given that the major deadenylase (Ccr4p) is not required for nonstop decay (see Example 1) and that degradation occurs by the exosome, it is possible that the exosome both deadenylates and degrades nonstop mRNAs. This would be surprising because normal mRNAs cannot be deadenylated by the exosome (5). Alternatively, an unidentified nuclease may remove the poly-adenylate [poly(A)] tail from nonstop mRNAs, followed by exosome-mediated decay.

To investigate whether the exosome degrades the poly(A) tail of nonstop transcripts, we performed transcriptional pulse-chase experiments. In these experiments, transcription of the reporter mRNA was induced briefly and was followed by transcriptional repression, which yielded a synchronous population of mRNA whose fate could be monitored. For comparison, wild-type PGK1 mRNA was synthesized with a poly(A) tail of approximately 70 residues and was subsequently deadenylated slowly (12). In contrast, nonstop-PGK1 transcripts disappeared rapidly without any detectable deadenylation intermediates. In addition, in a ski7Δ strain, the nonstop mRNA persisted as a fully polyadenylated species for 8 to 10 min before disappearing. These data indicate that exosome function is required for rapid degradation of both the poly(A) tail and the body of the mRNA. This data suggests that nonstop mRNAs are rapidly degraded in a 3′-to-5′ direction by the exosome, beginning at the 3′ end of the poly(A) tail (13). (Methods: Normal and nonstop PGK1 mRNAs were analyzed by a transcriptional pulse-chase experiment in wild-type strains and a ski7 deletion strain. The nonstop strains had the nonstop PGK1 gene on a plasmid and were grown to early- to mid-log phase at 24° C. in media containing 2% sucrose and lacking uracil. The strain shown normal was grown in 1% yeast, 2% peptone 2% sucrose media and carried the reporter integrated into the genome. However, similar results were obtained with a strain carrying PGK1 on a plasmid and grown in URA media. We turned on transcription for 8 min by replacing the media with media containing 2% galactose. We then terminated transcription by adding 4% glucose (T=0 min), and time points were taken. Forty micrograms of RNA isolated from each aliquot was cleaved with ribonuclease H (Promega, Madison, Wis.) using oRP70 (CGGATAAGAAAGCAACACCTGG (SEQ ID NO:2)) and analyzed by Northern blotting with a 6% polyacrylamide gel.)

Two observations suggest a mechanism by which nonstop mRNAs are specifically recognized and targeted for destruction by the exosome. First, nonstop mRNA degradation requires that a translating ribosome reach at least the poly(A) tail, and most likely the 3′ end of the mRNA (2, 14). The simplest interpretation of these data is that nonstop mRNAs are recognized when a ribosome reaches the 3′ end of the mRNA. Such a recognition would be analogous to the recognition of ribosomes with an empty A site by a tRNA-mRNA hybrid (tmRNA) in prokaryotes (15, 16). Second, the COOH-terminal region of the Ski7 protein is closely related to the guanosine triphosphatases (GTPases) EF1A and eRF3, including similarity in the GTPase domain (17-19). EF1A and eRF3 are translation factors that interact with the A site of the ribosome when it contains a sense or nonsense codon, respectively. The interaction of Ski7p homologs with the ribosomal A site suggests that the homologous domain of Ski7p may function to distinguish nonstop from normal mRNAs by binding to the empty A site of ribosomes that have reached the 3′ end of the mRNA. This hypothesis predicts that the COOH-terminal domain of Ski7p is specifically required for nonstop decay but may not be required for exosome-mediated degradation of normal mRNAs.

To determine the function of the Ski7p domains in exosome-mediated decay of nonstop and normal mRNAs, we generated yeast strains that express different deletion mutants of Ski7p (20). Two lines of evidence indicate that the NH₂-terminal nonconserved domain of Ski7p is necessary and sufficient for exosome-mediated degradation of normal mRNAs and that the COOH-terminal GTPase domain does not play a role in exosome-mediated degradation of normal mRNAs. First, the NH₂-terminal domain, but not the COOH-terminal domain, is required for viability under conditions in which exosome-mediated decay is essential for viability (19). Second, the deletion of the NH₂-terminal part, but not the COOH-terminal part, of Ski7p causes a dramatic decrease in the rate of exosome-mediated decay of normal mRNAs (19). Both ski7 alleles stabilized the nonstop reporter transcript, indicating that the COOH-terminal part of Ski7p functions in the nonstop mRNA degradation pathway. However, the COOH-terminal truncation of Ski7p has a smaller effect than either the NH₂-terminal deletion or complete deletion of SKI7. This suggests that other factors may to some extent be able to substitute for the COOH-terminal domain. Taken together, these results indicate that the NH₂-terminal part of Ski7p plays a central role in exosome-mediated mRNA decay and that the COOH-terminal domain plays a specific role in the degradation of nonstop mRNAs.

These results are consistent with the hypothesis that an interaction between the GTPase domain of Ski7p and the ribosome triggers exosome-mediated decay. One simple possibility is that Ski7p recruits the exosome to nonstop mRNAs. Consistent with this possibility, we observed that a large proportion of Ski7p copurified with two different subunits of the exosome (Ski4p or Rrp4p) (21, 22). Ski7p remained in the unbound fraction in control purifications from strains with an untagged exosome. These results indicate that Ski7p physically associates with the exosome. This association is specific because neither Ski3p nor Lsm1p copurified with the exosome (22). In addition, the copurification of Ski7p with both Ski4p and Rrp4p is resistant to washing with 1 M NaCl (22), suggesting a strong interaction between Ski7p and the exosome. The nuclear form of the exosome contains one additional subunit, Rrp6p (23, 24). Purification of protein A-tagged Rrp6 did not result in copurification of Ski7p (22), which is consistent with Ski7p being specific to the cytoplasmic exosome. Recently, Araki et al. (11) independently found that, when overexpressed, the NH₂-terminal part of Ski7p can coimmunoprecipitate with the exosome. The finding that Ski7p stably associates with the exosome through its NH₂-terminus suggests a mechanism to recruit the exosome to nonstop mRNAs recognized by the COOH-terminal of Ski7p. (Methods: Equal aliquots of each fraction of exosome purifications were analyzed by Western blotting using antibodies to protein A (Sigma) or to HA (Roche).)

To determine whether the interaction of Ski7p with the exosome is biologically relevant, we examined whether mutations in the exosome that disrupt all Ski7p-dependent functions of the exosome also disrupt Ski7-exosome interaction. The ski4-1 mutation severely reduced the copurification of Ski7p with the exosome. One possibility is that the amino acid change in ski4-1 changes the binding site for Ski7p. This same ski4-1 mutation blocked exosome-mediated decay of both nonstop and normal mRNAs (9). The observation that a mutation that prevents Ski7p from interacting with the exosome inhibits exosome-mediated mRNA decay indicates that the association of Ski7p with the exosome is important for the degradation of both normal and nonstop mRNAs.

One class of endogenous mRNAs subject to nonstop decay results from premature polyadenylation within the coding region (2). Another potential role for nonstop decay is to ensure the completeness of degradation for mRNAs that initiate 3′-to-5′ decay while still being translated. In this case, as the exosome enters the coding region from the 3′ end, it would encounter ribosomes coming from the 5′ end. In both cases, the reason for the rapid degradation of nonstop mRNAs would be to prevent the production of truncated proteins. Similarly, translation of aberrant mRNAs containing premature termination codons has previously been shown to be deleterious to Caenorhabditis elegans (25). To test whether nonstop mRNAs can be translated into protein, we generated a nonstop allele of the HIS3 gene. The nonstop his3 allele failed to complement a his3 deletion in a SKI+ strain. However, the nonstop his3 allele allowed rapid growth in the absence of added histidine when the strain was deleted for SKI2, SKI7, or SKI8. Even the COOH-terminal truncation of Ski7p, which specifically inhibits nonstop mRNA decay, allows for some growth in the absence of added histidine. These data suggest that the degradation of nonstop (his3) mRNA is effective in limiting the production of aberrant (His3p) protein, and in the absence of this mRNA degradation pathway, protein products of nonstop mRNAs accumulate to functional levels. (Methods: The HIS3 gene was amplified by polymerase chain reaction using oRP1075 (CGAGAGCTCAACACAGTCCTTTCCCGCAA (SEQ ID NO:3)) and oRP1077 (CGAGGATCCACTTGCCACCTATCACC (SEQ ID NO:4)) and was cloned as a Sac I-Bam HI fragment into the CEN URA3 plasmid pRS416 (30). The nonstop his3 allele was created by deleting the first nucleotide of the termination codon (Quick-change kit, Stratagene). This creates an open reading frame that extends past the previously mapped polyadenylation sites (31). The nonstop his3 plasmid was transformed into strains that were ura3Δ and his3Δ and were either SKI+, ski2Δ, ski7Δ, ski8Δ, or ski7-ΔC. URA+ transformants were selected and streaked onto a plate lacking histidine. The plate was then incubated for 2 days at 30° C.

In combination, these results define a mechanism of mRNA quality control that recognizes and degrades yeast mRNAs lacking translation codons, thereby preventing the production of truncated proteins. Because Ski protein homologs are present in the human genome (19, 26), the mechanism of nonstop decay is should be conserved. Transcripts that lack a termination codon are also recognized in prokaryotes (15, 16).

### REFERENCES AND NOTES

Numbers contained within parentheses in Example 1 (above) refer to the following notes and references. All of the following notes and references are incorporated herein by reference.

1. P. Hilleren, R. Parker, RNA 5, 711 (1999).

3. D. Muhlrad, C. J. Decker, R. Parker, Genes Dev. 8, 855 (1994).

4. C. Beelman et al., Nature 382, 577 (1996).

5. M. Tucker et al., Cell 104, 377 (2001).

6. J. S. Jacobs Anderson, R. Parker, EMBO J. 17, 1497 (1998).

7. A. van Hoof, R. Parker, Cell 99, 347 (1999).

8. P. Mitchell, D. Tollervey, Nature Struct. Biol. 7, 843 (2000).

9. A. van Hoof, R. R. Staples, R. E. Baker, R. Parker, Mol. Cell. Biol. 20, 8230 (2000).

10. J. Brown, X. Bai, A. W. Johnson, RNA 6, 449 (2000). 11. Y. Araki et al., EMBO J. 20, 4684 (2001).

12. D. Muhlrad, C. J. Decker, R. Parker, Mol. Cell. Biol. 15, 2145 (1995).

13. Nonstop mRNAs are not detectably deadenylated, even when they are stabilized by deletion of SKI7 or several other exosome mutations. One possible explanation is that a stalled ribosome occupies the extreme 3′ end of this mRNA and prevents exonucleases from digesting it. A corollary of this explanation is that in a wild-type strain, the exosome or associated proteins can dislodge a stalled ribosome at the 3′ end of the mRNA or initiate 3′-to-5′ decay of the mRNA in the presence of such a ribosome.

14. To test whether translation of nonstop PGK1 mRNA was required in cis, we introduced G₁₈ in its 5′ UTR. This sequence forms a stable secondary structure and reduces translation by 4 orders of magnitude (D. Muhlrad, C. J. Decker, R. Parker, Mol. Cell. Biol. 15, 2145 (1995)). This reduction in translation severely reduced exosome-mediated decay of the nonstop PGK1 mRNA (half-life 514 min).

15. A. W. Karzai, E. D. Roche, R. T. Sauer, Nature Struct. Biol. 7, 449 (2000).

16. A. Muto, C. Ushida, H. Himeno, Trends Biochem. Sci. 23, 25 (1998).

17. L. Benard, K. Carroll, R. C. Valle, D.C. Masison, R. B. Wickner, J. Virol. 73, 2893 (1999).

18. The SKI7 homology with translation factors is most evident in the GTPase domain, but multiple sequence alignment shows that the homology extends to the COOH-terminus of Ski7p, EF1A, and eRF3.

20. Alleles encoding either a COOH-terminal truncation or an NH₂-terminal deletion of Ski7p were integrated into the genome at the SKI7 locus and were expressed from the SKI7 promoter. The NH₂-terminal deletion removed amino acids 18 through 239, whereas the COOH-terminal truncation removed all amino acids from 265 to the COOH-terminal. The COOH-terminal truncation removes all of the translation factor homology.

21. Hemagglutinin (HA)-tagged Ski7p was generated as described in M. S. Longtine et al., Yeast 14, 953 (1998) and introduced into strains that carried a protein A-tagged version of Rrp4p (P. Mitchell, E. Petfalski, D. Tollervey, Genes Dev. 10, 502 (1996)), Ski4p, or Rrp6p, which are subunits of the exosome. As a control, we used a similarly HA-tagged version of Ski3p, which is known not to copurify with the exosome (J. Brown, X. Bai, A. W. Johnson, RNA 6, 449 (2000). 11. Y. Araki et al., EMBO J. 20, 4684 (2001)). All five tagged proteins are expressed from their normal genomic locus and are functional. Ski3p, Ski4p, Ski7p, and Rrp6 are also expressed from their own promoters, whereas Rrp4p is expressed from the GAL10 promoter (P. Mitchell, E. Petfalski, D. Tollervey, Genes Dev. 10, 502 (1996)). Protein extracts were prepared by vortexing in the presence of glass beads and 50 mM Tris-HCl (pH 7.5), 50 mM NaCl, 2 mM MgCl₂, 1 mM β-mercaptoethanol, 0.1% NP40, and complete protease inhibitors EDTA free (Roche, Basel, Switzerland) and were incubated at 4° C. for 1 hour with immunoglobulin G (IgG)-Sepharose beads. The beads were then washed twice with 40 volumes of the extraction buffer and twice with 40 volumes of the extraction buffer containing 1 M NaCl. The proteins bound to the IgG-Sepharose were recovered by boiling in sample buffer.

23. C. Allmang et al., Genes Dev. 13, 2148 (1999). 24. K. T. Burkard, J. S. Butler, Mol. Cell. Biol. 20, 604 (2000).

25. R. Pulak, P. Anderson, Genes Dev. 7, 1885 (1993). 26. C. -Y. Chen et al., Cell 107, 455 (2001).

27. D. Muhlrad, C. J. Decker, R. Parker, Mol. Cell. Biol. 15, 2145 (1995).

28. M. S. Longtine et al., Yeast 14, 953 (1998).

29. P. Mitchell, E. Petfalski, D. Tollervey, Genes Dev. 10, 502 (1996).

30. R. S. Sikorski, P. Hieter, Genetics 122, 19 (1989). 31. S. Mahadevan, T. R. Raghunand, S. Panicker, K. Struhl, Gene 190, 69 (1997).

### EXAMPLE 3

### Increased Readthrough Achieved by Gentamycin After Inhibition of Exosome Function Using RNAi

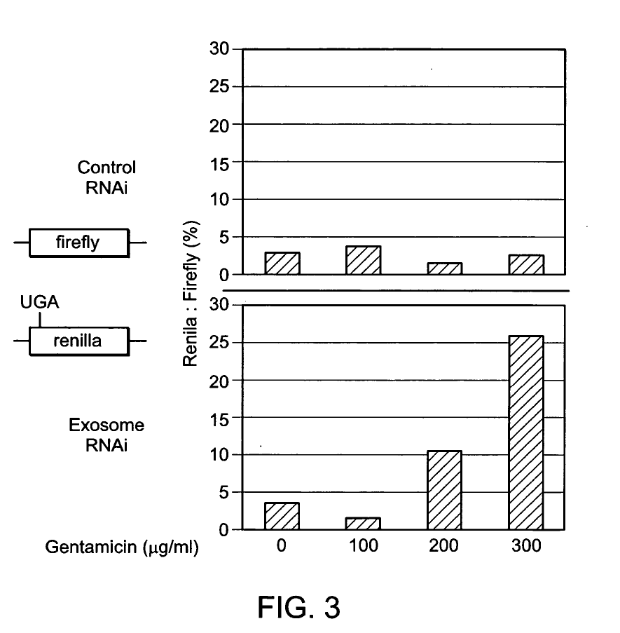

This example describes a readthrough assay that uses a dual lucifierase reporter construct. The firefly luciferase ORF is wild-type while translation of the renilla luciferase ORF is prevented by the presence of a termination codon (UGA). Cells were either untreated or treated with siRNAs directed against RRP4, an essential component of the exosome. With intact exosome function (control RNAi) increasing concentrations of gentamycin do not result in a significant increase in readthrough, as assessed by the renilla:firefly ratio (FIG. 3, top panel). Inhibition of exosome function results in a dose-dependent increase in readthrough attributable to gentamycin (FIG. 3, bottom panel).

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method of identifying a compound capable of inhibiting nonstop degradation of mRNA comprising:
a) contacting a cell comprising a reporter gene lacking a termination codon with a test compound;
b) measuring the level of expression or activity of the polypeptide encoded by the reporter gene; and
c) comparing the level of expression or activity of the polypeptide encoded by the reporter gene to the level of expression or activity of the polypeptide encoded by the reporter gene in control cells;
wherein a compound that upregulates the expression or activity of the polypeptide encoded by the reporter gene, as compared to the level of expression or activity of the polypeptide encoded by the reporter gene in control cells, is identified as a compound capable of inhibiting nonstop degradation of mRNA.

2. A method of identifying a compound capable of inhibiting nonstop degradation of mRNA comprising:
a) contacting a cell comprising a reporter gene that has a premature termination codon with a test compound;
b) contacting the cell with an agent that induces readthrough of premature termination codons;
c) measuring the level of expression or activity of the polypeptide encoded by the reporter gene; and
d) comparing the level of expression or activity of the polypeptide encoded by the reporter gene to the level of expression or activity of the polypeptide encoded by the reporter gene in control cells;
wherein a compound that upregulates the expression or activity of the polypeptide encoded by the reporter gene, as compared to the level of expression or activity of the polypeptide encoded by the reporter gene in control cells, is identified as a compound capable of inhibiting nonstop degradation of mRNA.

3. The method of claim 2, wherein the agent that induces readthrough of premature termination codons is an aminoglycoside antibiotic.

4. The method of claim 3, wherein the aminoglycoside antibiotic is selected from the group consisting of G-418, gentamycin, kanamycin, neomycin, netilmicin, paromomycin, streptomycin, tobramycin, hygromycin, amikacin, apramycin, and dihydrostreptomycin.

5. The method of any one of claims 1-2, wherein the reporter gene is contained within an expression vector.

6. The method of any one of claims 1-2, wherein the reporter gene encodes a protein selected from the group consisting of luciferase, β-galactosidase, chloramphenicol acetyl transferase, and a fluorescent protein.

7. The method of claim 6, wherein the luciferase is selected from the group consisting of firefly luciferase and Renilla luciferase.

8. The method of claim 6, wherein the fluorescent protein is selected from the group consisting of green fluorescent protein, enhanced green fluorescent protein, red fluorescent protein, yellow fluorescent protein, enhanced yellow fluorescent protein, blue fluorescent protein, and cyan fluorescent protein.

9. The method of any one of claims 1-2, wherein the cell is a eukaryotic cell.

10. The method of claim 9, wherein the cell is a yeast cell.

11. The method of claim 9, wherein the cell is a mammalian cell.

12. The method of claim 11, wherein the cell is a human cell.

13. The method of any of claims 1-2, wherein the level of expression of the polypeptide encoded by the reporter gene is measured by a method selected from the group consisting of Western blotting, ELISA, and RIA.

14. The method of claim 6, wherein the level of expression or activity of the polypeptide encoded by the reporter gene is determined by measuring an activity selected from the group consisting of luciferase activity, β-galactosidase activity, chloramphenicol acetyl transferase activity, and the level of fluorescence of the fluorescent protein.

15. The method of claim 14, wherein the activity is measured using and assay selected from the group consisting of a standard luciferase assay, a standard β-galactosidase assay, and a standard chloramphenicol acetyl transferase assay.

16. The method of any of claims 1-2, further comprising:
a) contacting a cell comprising a reporter gene lacking a termination codon with a test compound identified by the methods of any one of claims 1-2 as a compound capable of inhibiting nonstop degradation of mRNA;
b) measuring the half life of the reporter gene mRNA; and
c) comparing the half life of the reporter gene mRNA to the half life of the reporter gene mRNA in control cells,
wherein a compound that increases the half life of the reporter gene mRNA, as compared to the half life of the reporter gene mRNA in control cells, is confirmed as a compound capable of inhibiting nonstop degradation of mRNA.

17. The method of any of claims 1-2, further comprising:
a) contacting a cell comprising a reporter gene that has a premature termination codon with a test compound identified by the methods of any one of claims 1-2 as a compound capable of inhibiting nonstop degradation of mRNA;
b) contacting the cell with an agent that induces readthrough of premature termination codons;
c) measuring the half life of the reporter gene mRNA; and
d) comparing the half life of the reporter gene mRNA to the half life of the reporter gene mRNA in control cells,
wherein a compound that increases the half life of the reporter gene mRNA, as compared to the half life of the reporter gene mRNA in control cells, is confirmed as a compound capable of inhibiting nonstop degradation of mRNA.

18. The method of claim 17, wherein the agent that induces readthrough of premature termination codons is an aminoglycoside antibiotic.

19. The method of claim 18, wherein the aminoglycoside antibiotic is selected from the group consisting of G-418, gentamycin, kanamycin, neomycin, netilmicin, paromomycin, streptomycin, tobramycin, hygromycin, amikacin, apramycin, and dihydrostreptomycin.

20. The method of any one of claims 16-17, wherein the reporter gene is contained within an expression vector.

21. The method of any one of claims 16-17, wherein the reporter gene encodes a protein selected from the group consisting of luciferase, β-galactosidase, chloramphenicol acetyl transferase, and a fluorescent protein.

22. The method of claim 21, wherein the luciferase is selected from the group consisting of firefly luciferase and Renilla luciferase.

23. The method of claim 21, wherein the fluorescent protein is selected from the group consisting of green fluorescent protein, enhanced green fluorescent protein, red fluorescent protein, yellow fluorescent protein, enhanced yellow fluorescent protein, blue fluorescent protein, and cyan fluorescent protein.

24. The method of any one of claims 16-17, wherein the cell is a eukaryotic cell.

25. The method of claim 24, wherein the cell is a yeast cell.

26. The method of claim 24, wherein the cell is a mammalian cell.

27. The method of claim 26, wherein the cell is a human cell

28. The method of any one of claims 16-17, wherein the level of expression of the reporter gene mRNA is measured by a method selected from the group consisting of Northern blotting, primer extension, nuclease protection, and RT-PCR.

29. A method of treating a genetic disorder in a subject caused by a premature termination codon comprising administering to the subject a therapeutically effective amount of compound that induces readthrough of premature termination codons and a therapeutically effective amount of a compound that inhibits nonstop degradation of mRNA, thereby treating the genetic disorder in the subject.

30. The method of claim 29, wherein the disorder is selected from the group consisting of muscular dystrophy, cystic fibrosis, sever combined immune deficiency, and Hurler's syndrome.

31. The method of claim 29, wherein the agent that induces readthrough of premature termination codons is an aminoglycoside antibiotic.

32. The method of claim 31, wherein the aminoglycoside antibiotic is selected from the group consisting of G-418, gentamycin, kanamycin, neomycin, netilmicin, paromomycin, streptomycin, tobramycin, hygromycin, amikacin, apramycin, and dihydrostreptomycin.

33. The method of claim 29, wherein the compound that inhibits nonstop degradation of mRNA inhibits expression of at least one exosome protein or exosome associated protein.

34. The method of claim 29, wherein the compound that inhibits nonstop degradation of mRNA is an siRNA.


SEQUENCE LISTING (4 sequences)

SEQ ID NO: 1 - DNA, 27 bp, Saccharomyces cerevisiae
ctcggtcctgtaccgaacgagacgagg

SEQ ID NO: 2 - DNA, 22 bp, Saccharomyces cerevisiae
cggataagaaagcaacacctgg

SEQ ID NO: 3 - DNA, 29 bp, Saccharomyces cerevisiae
cgagagctcaacacagtcctttcccgcaa

SEQ ID NO: 4 - DNA, 26 bp, Saccharomyces cerevisiae
cgaggatccacttgccacctatcacc